(19) United States
(12) Patent Application Publication
(10) Pub. No.: US 2021/0369889 A1
(43) Pub. Date: Dec. 2, 2021

(54) PLAYING CARD CLEANER

(71) Applicant: SG Gaming, Inc., Las Vegas, NV (US)

(72) Inventors: Georg FEKETE, Korneuburg (AT); Peter KRENN, Neufeld (AT); David KRIENDLHOFER, Pressbaum (AT)

(21) Appl. No.: 17/202,808

(22) Filed: Mar. 16, 2021

Related U.S. Application Data
(60) Provisional application No. 63/029,871, filed on May 26, 2020.

(51) Int. Cl. A61L 2/10; A63F 1/06

(52) U.S. Cl. CPC A61L 2/10; A61L 2202/11; A63F 1/06

(57) ABSTRACT

A card cleaning apparatus comprises an input bin, an output bin, and a cleaning module disposed between the input and output bins. The input bin receives a stack of playing cards. The cleaning module includes a housing, a UV-C light source, and a card guide. The UV-C light source and the card guide are contained within the housing. A plurality of rollers transport individual cards from the stack along a card transport path. The card transport path extends from the input bin to the output bin via the card guide of the cleaning module. The UV-C light source emits UV-C light onto each card while it is in the card guide to clean the cards.

### CROSS-REFERENCE To RELATED APPLICATIONS

This application claims the benefit of priority to U.S. Provisional Application No. 63/029,871, filed May 26, 2020, the contents of which are hereby incorporated by reference in their entirety.

### COPYRIGHT

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever. Copyright 2021 SG Gaming, Inc.

### FIELD OF THE INVENTION

The present invention relates generally to apparatus and methods for handling playing cards and, more particularly, relates to apparatus and methods for cleaning playing cards.

### BACKGROUND OF THE INVENTION

Physical playing cards are used to play such casino table games as poker, blackjack, baccarat, Pai Gow, and various specialty card games. As the cards are handled by a dealer and/or players at the table, the cards may pick up, carry, and spread germs such as bacteria, viruses, and mold spores. As the same playing cards are re-used over multiple plays of a game, the germs on the cards may infect other individuals by cross-contamination with potentially harmful effects.

A need therefore exists for apparatus and methods for periodically cleaning used playing cards to minimize the transmission of germs between individuals via the cards.

### SUMMARY OF THE INVENTION

According to an embodiment of the present invention, a card cleaning apparatus comprises an input bin, an output bin, and a cleaning module disposed between the input and output bins. The input bin receives a stack of playing cards. The cleaning module includes a housing, a UV-C light source, and a card guide. The UV-C light source and the card guide are contained within the housing. A plurality of rollers transport individual cards from the stack along a card transport path. The card transport path extends from the input bin to the output bin via the card guide of the cleaning module. The UV-C light source emits UV-C light onto each card while it is in the card guide to clean the cards. The housing is sized to contain an entirety of each card while it is in the card guide. A method of cleaning playing cards with the card cleaning apparatus is also disclosed herein.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1 is an isometric view of a card cleaning apparatus according to an embodiment of the present invention.

FIG. 2 is a simplified and schematically illustrated cross-sectional view taken through the card cleaning apparatus along a plane parallel to the left and right sides of the apparatus (and perpendicular to the front and back sides of the card cleaning apparatus), wherein various components and features of the apparatus have been removed to facilitate illustration and description of operation of the apparatus.

FIG. 3A is a simplified isometric view of an interior of the card cleaning apparatus with a playing card disposed in a card cleaning module of the apparatus, wherein various components and features of the apparatus have been removed to facilitate illustration and description of operation of the apparatus.

FIG. 3B is a simplified isometric view of an interior of the card cleaning apparatus without a playing card disposed in the card cleaning module of the apparatus, wherein various components and features of the apparatus have been removed to facilitate illustration and description of operation of the apparatus.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.”

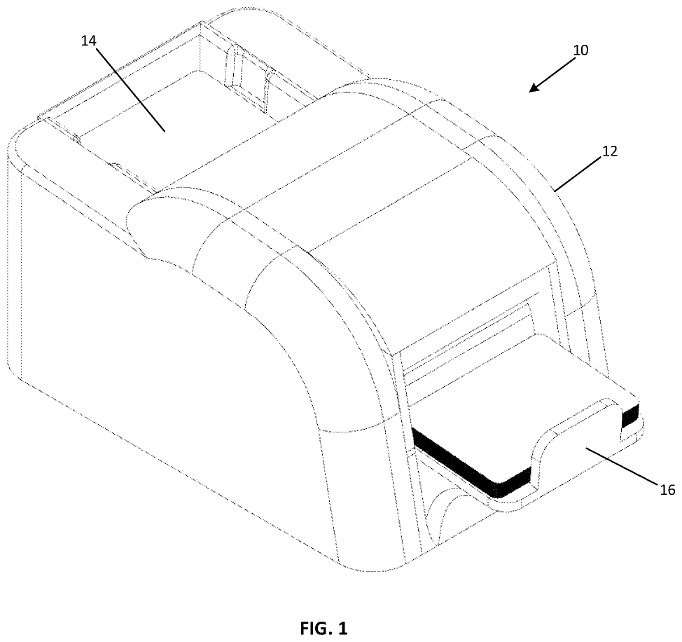

FIG. 1 illustrates a card cleaning apparatus 10 according to an embodiment of the present invention. The card cleaning apparatus 10 may be placed on a casino gaming table proximate a dealer and such other card handling devices as an automatic shuffler and a card shoe. The card cleaning apparatus 10 is used to periodically clean (sanitize or disinfect) used playing cards to minimize the transmission of germs between individuals via the cards. For example, the card cleaning apparatus 10 may clean playing cards before they can be touched by multiple different individuals (e.g., different players, different dealers, or a player and a dealer). The card cleaning apparatus 10 includes an exterior housing or enclosure 12, an input bin 14, and an output bin 16. These components may be composed of molded plastic. The input bin 14 receives a stack of playing cards, e.g., used cards or a full deck of cards, to be input into the card cleaning apparatus 10. The output bin 16 receives playing cards after they have been cleaned by the card cleaning apparatus 10.

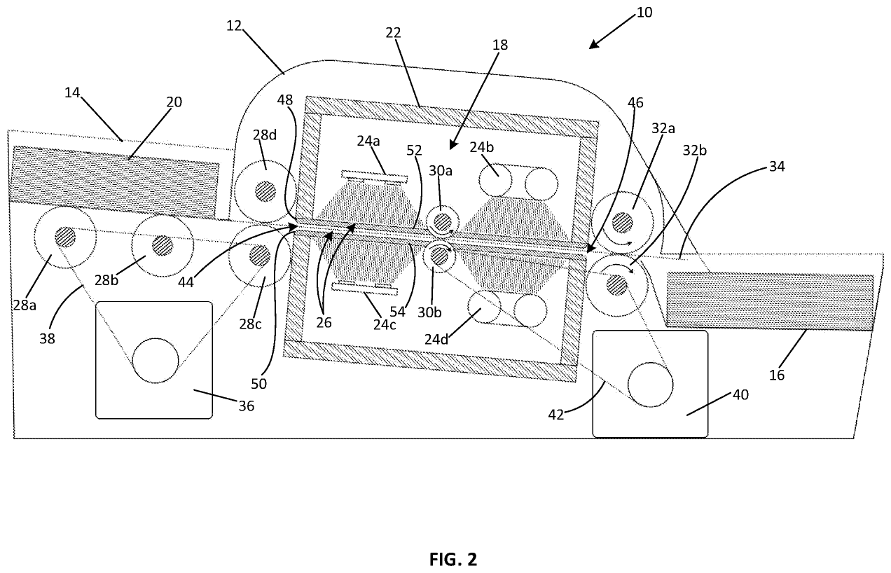

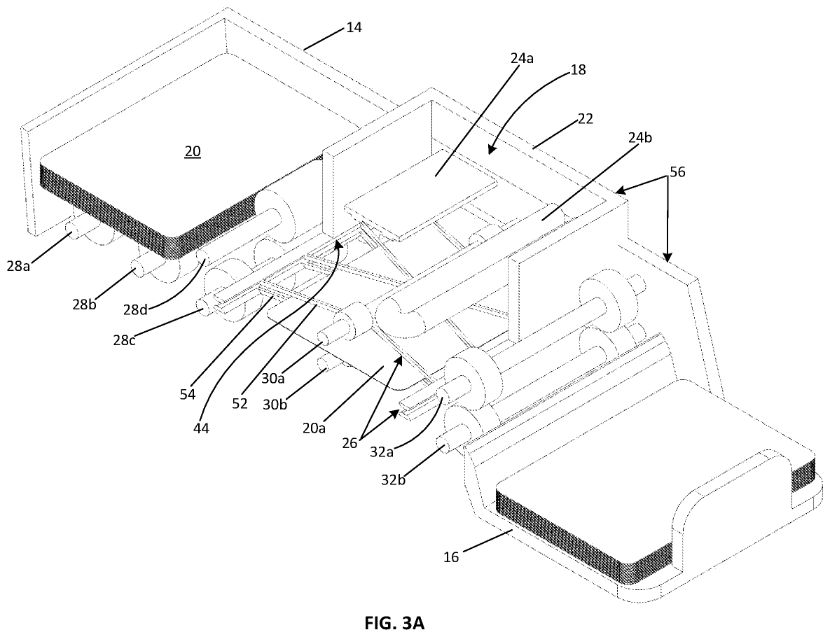

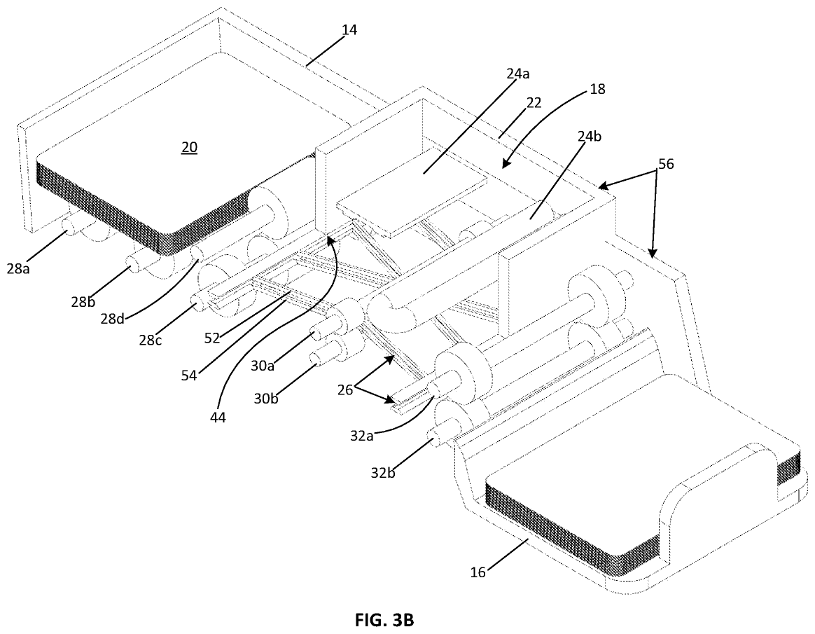

Referring to FIGS. 2, 3A, and 3B, the card cleaning apparatus 10 comprises the input bin 14, the output bin 16, and a cleaning module 18 disposed between the input bin 14 and the output bin 16. The input bin 14 includes a pair of opposing side walls (only the far side wall is shown in FIGS. 3A and 3B to facilitate viewing of other components) and a back wall bridging the opposing side walls. The input bin 14 has no front wall opposing its back wall and is open at its top and bottom. The input bin 14 receives a stack 20 of playing cards. The cleaning module 18 includes a housing 22, a UV-C light source 24a-d (see FIG. 2 for UV-C light source elements 24c-d), and a card guide 26. The UV-C light source 24a-d and the card guide 26 are contained within the housing 22 (which is only shown in part in FIGS. 3A and 3B to facilitate viewing of other components). A plurality of rollers 28a-d, 30a-b, and 32a-b transport individual playing cards from the stack 20 along a card transport path 34 (see FIG. 2). The card transport path 34 extends from the input bin 14 to the output bin 16 via the card guide 26 of the cleaning module 18. A pair of opposing vertical interior side walls span the length of the card cleaning apparatus to laterally retain playing cards in the card transport path 34. To facilitate viewing of other components, FIGS. 3A and 3B only depict the far interior side wall 56. The UV-C light source 24a-d emits UV-C light onto each card 20a (see FIG. 3A) while it is in the card guide 26 to clean the cards.

The plurality of rollers include a first set of transport rollers 28a-d, a second set of transport rollers 30a-b, and a third set of transport rollers 32a-b. The first set of transport rollers 28a-d transport a card in the stack 20 from the input bin 14 to the card guide 26. The rollers 28a-d are preferably composed of a rubber material such as urethane. The rollers 28a-c are linked to a shaft of a motor 36 (see FIG. 2) by a belt 38 (see FIG. 2) and rotated at a common speed. The roller 28d is a brake roller preferably composed of stainless steel or aluminum and is not driven by the motor 36. The motor-driven rollers 28a-b (also known as pick-off rollers) are disposed beneath the stack 20 of cards in the input bin 14. The rollers 28a-b draw a bottommost card from the stack 20 and move that card towards the card guide 26 while the brake roller 28d retains (blocks forward motion of) the remaining cards in the stack 20 in the input bin 14. The brake roller 28d has a clutch bearing that permits reverse motion in the event any cards get jammed in the apparatus and need to be returned to the input bin 14. When a leading edge of the card reaches the rollers 28c-d, the motor-driven roller 28c moves the card into the card guide 26.

The second set of transport rollers 30a-b transport the card 20a (see FIG. 3A) through the card guide 26 and are centrally located between the front (upstream) and back (downstream) ends of the card guide 26. The rollers 30a-b are preferably composed of stainless steel, aluminum, or other UV-C resistant material to minimize any degradation that may otherwise be caused by the UV-C light emitted by the UV-C light source 24a-d. The third set of transport rollers 32a-b (also known as output rollers) transport the card from the card guide 26 to the output bin 16. The rollers 32a-b are preferably composed of a rubber material such as urethane. The rollers 30a-b and 32a-b are coupled to another motor 40 (see FIG. 2) and rotated at a common speed that is higher than the speed of rotation of the rollers 28a-c in order to create a gap or separation between adjacent ones of the series of cards transported along the card transport path 34. In the illustrated embodiment, the motor 40 is linked to the bottom rollers 30b and 32b by a belt 42 (see FIG. 2), and the bottom rollers 30b and 32b, in turn, drive the respective top rollers 30a and 32a via linked gears. The top or bottom rollers in the apparatus 10 may be spring loaded towards the respective opposing rollers to apply pressure to the playing cards as they are individually transported through the apparatus 10. The distance between any adjacent pairs of top/bottom rollers (e.g., between adjacent roller pairs 28c-d and 30a-b and between adjacent roller pairs 30a-b and 32a-b) is less than the width of a playing card so that each pair of top/bottom rollers is able to push the card to the next pair of top/bottom rollers along the card transport path 34. For example, if a card width ranges from 57 to 63 millimeters, then the distance between adjacent roller pairs is about 55 millimeters or less.

The housing 22 includes an entrance 44 and an exit 46 (see FIG. 2) and effectively contains the UV-C light from the UV-C light source 24a-d within the cleaning module 18. An interior surface of the housing 22 may be coated with a reflective material. The card guide 26 extends between the entrance 44 and the exit 46. The card guide 26 includes a pair of opposing entrance edges 48 and 50 (see FIG. 2) at the entrance 44 that are on opposite sides of the card transport path 34 and are beveled or curved as shown to guide the individual playing cards into the card guide 26. To facilitate entry into the card guide 26 of a playing card that typically has a thickness of about 0.3 millimeters but may be bent, frayed, or torn from prior use, the opposing entrance edges 48 and 50 at the entrance 44 may have a minimum gap therebetween of approximately 3 millimeters, i.e., the card transport path 34 at the entrance 44 may have a height of approximately 3 millimeters. In the illustrated embodiment, the card guide 26 includes a pair of opposing metal grids 52 and 54 on opposite sides of the card transport path 34. The metal grids are preferably composed of stainless steel, aluminum, or other UV-C resistant material to minimize any degradation that may otherwise be caused by the UV-C light emitted by the UV-C light source 24a-d. The metal grids 52 and 54 and transport rollers 30a-b only block a small percentage (e.g., 12.5 percent) of the UV-C light from the UV-C light source 24a-d. And the bars of the metal grids 52 and 54 are angled (i.e., not parallel to the card transport path 34) so that the metal grids do not always cover the same parts of the playing card 20a as it passes through the card guide 26. The entire playing card 20a thus receives exposure to the UV-C light. In an alternative embodiment, the card guide 26 includes a pair of opposing transparent quartz glass walls on opposite sides of the card transport path 34.

The card transport path 34 may be angled downward relative to the horizontal by approximately 5 degrees (see FIG. 2). This downward angle reduces the footprint of the card cleaning apparatus 10 and enables gravity to assist in drawing cards from the input bin 14 and dropping cleaned cards into the output bin 16.

The UV-C light source 24a-d is operated to kill at least a 90 percent of germs on each playing card, or more preferably 99 percent of germs, or most preferably 99.9 percent of germs. In one embodiment, each playing card receives a UV-C energy dose (i.e., total accumulative exposure of UV-C received) from the UV-C light source 24a-d of at least about 30 millijoules per square centimeter (mJ/cm²). The UV-C light source 24a-d emits ultraviolet light with wavelengths between 200 and 280 nanometers (nm). In one embodiment, the UV-C light source 24a-d includes at least one of a DC-powered light-emitting diode (LED) that emits ultraviolet light with wavelengths in the 260-280 nm range or an AC-powered “mercury-vapor” lamp that emits ultraviolet light with wavelengths near 254 nm. Although the figures illustrate both types of light sources, it is preferable to use just one type of light source in the cleaning module 18. In one embodiment, the UV-C light source 24a-d includes a pair of UV-C light source elements 24a-b disposed above the metal grid 52 and a pair of UV-C light source elements 24c-d (see FIG. 2) disposed below the metal grid 54. The number and arrangement of UV-C light source elements within the housing 22 may be varied to deliver the desired UV-C energy dose to each playing card. While the playing card 20a is transported through the card guide 26 and the entire card 20a (see FIG. 3A) is contained within the housing 22, the elements 24a-b and 24c-d emit UV-C light onto respective opposite surfaces of the playing card 20a. By adjusting the rotational speeds of the transport rollers, the speed of movement of the playing cards through the card cleaning apparatus 10 may be adjusted to increase or decrease the amount of exposure of each card to the UV-C light, thereby changing the extent to which the UV-C light kills any germs on the cards.

The card cleaning apparatus 10 may include a variety of sensors. For example, optical or mechanical sensors may be disposed at regular intervals along the card transport path 34 to track and count the playing cards as they are individually transported through the apparatus. An ultraviolet sensor may measure the UV-C radiation emitted by the UV-C light source 24a-d to ensure the playing cards are effectively cleaned. While the card cleaning apparatus 10 is cleaning a stack 20 of cards, the UV-C light source 24a-d is powered on and continuously emitting UV-C light. However, a security switch may detect opening of a cover, case, lid, or door of the card cleaning apparatus 10 and, in turn, cause the UV-C light source 24a-d to automatically switch or stay off when such opening occurs.

The card cleaning apparatus 10 may be operated to clean a standard deck of 52 playing cards in about one minute and, more preferably, in about 30 seconds. For example, if the apparatus 10 is continuously operated (i.e., the transport rollers are in continuous motion and the cards in the stack 20 are continuously drawn and individually transported in series through the apparatus 10), the cards may be transported through the apparatus 10 with each card exposed to the UV-C light in the cleaning module 18 for about 1 second and, more preferably, about 0.5 seconds. To minimize the amount of time spent on cleaning playing cards and maximize the number of games played at a casino gaming table, the card cleaning apparatus 10 may be used to clean only the playing cards that are touched by players. By way of example, in standard Texas Hold ‘Em poker, the dealer deals two cards to each player and five community cards from a standard deck of 52 cards. If the table is occupied by seven players and only the players’ cards are cleaned, then the duration of the cleaning process can be reduced by about 73%, i.e., 100−(100×14/52). If the card cleaning apparatus 10 is used to also clean the five community cards touched by the dealer, then the duration of the cleaning process is still reduced by about 63%. Accordingly, after the dealer deals a hand/round of a card game, the dealer collects the used cards and inserts them into the card cleaning apparatus 10. The remaining cards (i.e., the unused cards that were not in play in the prior hand/round of the card game) may bypass the card cleaning apparatus 10. After the used cards are cleaned and deposited in the output bin 16 of the card cleaning apparatus 10, they are combined with the unused cards (which bypassed the card cleaning apparatus) and inserted into and shuffled by an automatic card shuffler, which may be positioned proximate the card cleaning apparatus 10 on the gaming table. The shuffled cards are then ready for use in a subsequent hand/round of the card game.

The card cleaning apparatus 10, particularly such interior components as the transport rollers 28a-d, 30a-b, and 32a-b and the card guide 26, may accumulate germs, dust, and dirt over time. To clean such components, the card cleaning apparatus 10 may be periodically operated in a “device cleaning mode” (e.g., on every dealer shift change) in which one or more cleansing cards are run through the apparatus 10. The cleansing cards have the same size as a standard playing card and may be soaked or coated with alcohol, copper/silver ion, and/or other anti-germ treatment. Different cleansing cards may bear different treatments. The device cleaning mode may reduce the amount of work otherwise performed by a service technician every few weeks.

The cleaning module 18 may be divided into first and second distinct modules stacked on top of each other. Each module may contain its own UV-C light source on only one side of the card transport path. The first module may clean a top surface of a playing card. After a mechanism flips the card over after it leaves the first module and before it enters the second module, the second module may clean a bottom surface of the card.

The cleaning module 18 may be part of a standalone card cleaning apparatus 10 as shown and described, or may be integrated along a card transport path in other card handling devices such as a deck checker, an automatic card shuffler, or a card shoe of the types disclosed in U.S. Patent Nos. 6,726,205, 9,566,501, 8,960,674, 8,628,086, and 7,556,266, each of which is incorporated herein by reference in its entirety.

Each of these embodiments and obvious variations thereof is contemplated as falling within the spirit and scope of the claimed invention, which is set forth in the following claims. Moreover, the present concepts expressly include any and all combinations and subcombinations of the preceding elements and aspects.

What is claimed is:

1. A card cleaning apparatus, comprising:
an input bin configured to receive a stack of playing cards;
an output bin;
a cleaning module disposed between the input bin and the output bin, the cleaning module including a housing, a UV-C light source, and a card guide, the UV-C light source and the card guide being contained within the housing; and
a plurality of rollers configured to transport individual cards from the stack along a card transport path, the card transport path extending from the input bin to the output bin via the card guide of the cleaning module, the UV-C light source configured to emit UV-C light onto each card while it is in the card guide, the housing being sized to contain an entirety of each card while it is in the card guide.

2. The apparatus of claim 1, wherein the plurality of rollers include a first set of transport rollers, a second set of transport rollers, and a third set of transport rollers, the first set of transport rollers configured to transport a card in the stack from the input bin to the card guide, the second set of transport rollers configured to transport the card through the card guide, and the third set of transport rollers configured to transport the card from the card guide to the output bin.

3. The apparatus of claim 2, wherein the first set of transport rollers includes a pick-up roller disposed beneath the stack of playing cards in the input bin, the pick-up roller configured to transport a bottom card in the stack from the input bin towards the card guide.

4. The apparatus of claim 2, wherein the second set of transport rollers are driven at a higher speed than the first set of transport rollers to create a gap between adjacent ones of the individual cards.

5. The apparatus of claim 2, wherein the second set of transport rollers are composed of stainless steel or aluminum.

6. The apparatus of claim 1, wherein the card transport path is angled downward relative to the horizontal by approximately 5 degrees.

7. The apparatus of claim 1, wherein the UV-C light source includes at least one of a DC-powered light-emitting diode (LED) or an AC-powered lamp.

8. The apparatus of claim 1, wherein the UV-C light source includes first and second UV-C light source elements that are on opposite sides of the card transport path and are configured to emit the UV-C light onto respective opposite surfaces of each card.

9. The apparatus of claim 1, wherein the card transport path within the card guide has a height of approximately 3 millimeters.

10. The apparatus of claim 1, wherein the card guide includes a pair of opposing entrance edges that are on opposite sides of the card transport path and are beveled or curved to guide the individual playing cards into the card guide.

11. The apparatus of claim 1, wherein the card guide includes a pair of opposing UV-C resistant members on opposite sides of the card transport path, the UV-C resistant members being at least one of metal grids or glass walls.

12. The apparatus of claim 1, wherein the apparatus is independent of any shuffler for shuffling cards and any shoe for dealing cards onto a table.

13. A card cleaning apparatus, comprising:
a housing including an entrance and an exit;
a card guide contained within the housing and extending between the entrance and the exit;
a plurality of rollers configured to transport individual playing cards from the entrance to the exit, via the card guide, along a card transport path; and
a UV-C light source contained within the housing and configured to emit UV-C light onto each card while it is in the card guide, the housing being sized to contain an entirety of each card while it is in the card guide.

14. The apparatus of claim 13, wherein the plurality of rollers include a set of rollers composed of stainless steel or aluminum.

15. The apparatus of claim 13, wherein the card transport path is angled downward relative to the horizontal by approximately 5 degrees.

16. The apparatus of claim 13, wherein the UV-C light source includes at least one of a DC-powered light-emitting diode (LED) or an AC-powered lamp.

17. The apparatus of claim 13, wherein the UV-C light source includes first and second UV-C light source elements that are on opposite sides of the card transport path and are configured to emit the UV-C light onto respective opposite surfaces of each card.

18. The apparatus of claim 13, wherein the card transport path within the card guide has a height of approximately 3 millimeters.

19. The apparatus of claim 13, wherein the card guide includes a pair of opposing entrance edges at the entrance that are on opposite sides of the card transport path and are beveled or curved to guide the individual playing cards into the card guide.

20. The apparatus of claim 13, wherein the card guide includes a pair of opposing UV-C resistant members on opposite sides of the card transport path, the UV-C resistant members being at least one of metal grids or glass walls.

21. The apparatus of claim 13, wherein the apparatus is independent of any shuffler for shuffling cards and any shoe for dealing cards onto a table.

22. A method of cleaning playing cards, comprising:
receiving a stack of playing cards in an input bin;
transporting, with a plurality of rollers, individual cards from the stack along a card transport path, the card transport path extending from the input bin to an output bin via a card guide, the card guide being contained within a housing; and
emitting, by a UV-C light source, UV-C light onto each card while it is in the card guide, the housing being sized to contain an entirety of each card while it is in the card guide.

23. The method of claim 22, wherein the plurality of rollers include a first set of transport rollers, a second set of transport rollers, and a third set of transport rollers, and wherein the transporting includes:
transporting, with the first set of transport rollers, a card in the stack from the input bin to the card guide;
transporting, with the second set of transport rollers, the card through the card guide; and
transporting, with the third set of transport rollers, the card from the card guide to the output bin.

24. The method of claim 22, wherein the UV-C light source includes at least one of a DC-powered light-emitting diode (LED) or an AC-powered lamp.

25. The method of claim 22, wherein the UV-C light source includes first and second UV-C light source elements that are on opposite sides of the card transport path, and wherein the emitting includes emitting the UV-C light onto respective opposite surfaces of each card.

26. The method of claim 22, wherein the card guide includes a pair of opposing UV-C resistant members on opposite sides of the card transport path, the UV-C resistant members being at least one of metal grids or glass walls.

27. The method of claim 22, wherein the input bin, the output bin, the card guide, the housing, the plurality of rollers, and the UV-C light source are integrated in an apparatus that is independent of any shuffler for shuffling cards and any shoe for dealing cards onto a table.
